(19) United States
(12) Patent Application Publication
(10) Pub. No.: US 2021/0290742 A1
(43) Pub. Date: Sep. 23, 2021

(54) METHODS OF USING LYSINE DEACETYLASE (KDAC) INHIBITION TO GENERATE ANTIGEN SPECIFIC MEMORY T CELL RESPONSES FOR DURABLE IMMUNOTHERAPY

(71) Applicant: ARIZONA BOARD OF REGENTS ON BEHALF OF THE UNIVERSITY OF ARIZONA, Tucson, AZ (US)

(72) Inventor: Protul A Shrikant, Tucson, AZ (US)

(21) Appl. No.: 17/268,806

(22) PCT Filed: Sep. 27, 2019
(86) PCT No.: PCT/US19/53550

§ 371 Date: Feb. 16, 2021

Related U.S. Application Data
(60) Provisional application No. 62/737,707, filed on Sep. 27, 2018.

(51) Int. Cl. A61K 39/00; C12N 5/0783

(52) U.S. Cl. CPC A61K 39/0011; A61K 39/0008; C12N 5/0636; A61K 2039/5158; C12N 2501/2321; C12N 2501/2301; C12N 2501/2302; C12N 2501/2312; C12N 2501/73

(57) ABSTRACT

A method is described herein for generating antigen-specific memory T. cells for effective immunotherapy responses using pan inhibitors of lysine deacetylase (KDAC), The present invention features the introduction of pan KDAC inhibitors during T-cell culture and/or vaccination to tune T cell differentiation into memory T cells for persistent antigen-specific responses. The current invention can be applied to the generation of personalized immunotherapies, including: 1) durable immunotherapy generation for the pharmaceutical industry; 2) patient-specific immunotherapy tor personalized medicine; and 3) specific memory T cell population generation or T cell therapy for cancer and/or infections for personalized cancer immunotherapy. The present invention relates to a method to induce acquired T cell differentiation towards the generation of specific memory T cells with selective functions for treatment.

### CROSS-REFERENCES TO RELATED APPLICATIONS

This application claims benefit of U.S. Patent Application No. 62/737,707 filed Sep. 27, 2018, the specification(s) of which is/are incorporated herein in their entirety by reference.

### BACKGROUND OF THE INVENTION

### Field of the Invention

The present invention relates to methods to produce durable responses to immunotherapy by inducing T cell differentiation towards the generation of antigen-specific T cells with selective functional phenotypes. The tuned T cells are then used for treatment of chronic challenges such as cancer and/or infections. In particular, the present invention features a method for using lysine deacetylase inhibitors including histone deacetylase inhibitors) curing T-cell culture and/or vaccination to tune their differentiation into memory T cells for persistent antigen-specific responses. The memory cells are characterized by their cell surface markers, metabolic profile, transcription/signaling profile, and their functional phenotype. This method is useful to generate a specific memory T cell population that is effective in T cell therapy of chronic challenges such as cancer and/or infections.

### Background Art

The favorable outcomes achieved by immunotherapies including Chimeric Antigen Receptor (CAR) therapy and check-point blockade therapy encourage new approaches for T cell therapy of chronic challenges such as cancer and/or viral infections. The Therapeutic index (efficacy versus side effects) in the clinic and preclinical animal models is determined by persistence of the induced (active) and/or adoptively transferred (passive) T cells as well as the extent of toxicity produced due to overt effector functions (cytokine release and/or auto-reactivity). Memory T cell responses are persistent and demonstrate recall to antigen specific responses in a regulated manner that are ideally suited for T cell therapies for chronic challenges.

### BRIEF SUMMARY OF THE INVENTION

The present invention features a method for using KDAC inhibition (e.g., using a pan lysine de-acetylase inhibitor (KDACi; e.g., Trichostatin A: TSA) during T-cell culture and/or vaccination to tune their differentiation into memory T cells for persistent antigen specific responses. The present invention incorporates deacetylase inhibitors during T-cell culture to stimulate differentiation into memory T cells for durable immunotherapy responses. The memory cells are characterized by their cell surface phenotype, metabolic profile, transcription/signaling profile, and their functional phenotype. This method is useful to generate antigen-specific T cell populations with memory function that is likely more effective in T cell therapy of chronic challenges such as cancer and/or infections.

One of the unique and inventive technical features of the present invention is that during the generation of T cells for adoptive T cell therapy (e.g., CAR/TCR), a pan KDACi is introduced early to tune T cell differentiation into memory T cells for persistent antigen-specific responses. While pan-KDAC inhibitors directly impact tumor growth, their broad targeting can be detrimental to the immune system. It was surprising then that pan KDAC inhibition differentially regulates antigen induced early T cell activation phenotype; for example, by selectively restricting antigen stimulation induced CD69 transcription but enhancing CD62L (L-selectin) shedding.

The incorporation of KDAC inhibitors at different amounts d at different tunes and for e durations of T-cell culture, including pre- and post-antigen stimulation allows T cells to be differentiated towards distinct functional phenotypes. Without wishing to limit the invention to any theory or mechanism, it is believed that the technical feature of the present invention, perturbations of KDAC inhibition, advantageously provides for differentiation of T cells tuned for specific functional phenotypes, in particular memory T cells. For example, early. KDAC inhibition of antigen stimulated T cells produces memory T cells for persistent antigen-specific responses. None of the presently known prior references or work has the unique inventive technical feature of the present invention. In addition, the present invention allows inhibitors of specific KDAC isoforms (e.g., KDAC1, KDAC2, KDAC6, KDAC11, etc.) to have distinct ability to regulate T cell functional differentiation, and thus can be used to produce a variety of antigen specific functional CD8+ T cells for therapy.

The present invention features an in vitro method of tuning or differentiating T cells to generate a population of T cells for effective and durable immunotherapy responses, preferred embodiments, the method comprises first culturing T cells obtained from a source (e.g., a source can be a human or cultured cells) and stimulating the T cells in culture with an antigen [e.g., major histocompatibility complex (MHC) Class I; HLA-A, HLA-B, HA-C], co-stimulatory molecules (e.g., 87-related family members or TNF-related family members), cytokines [e.g., interleukin (IL)-1 IL-2, IL-12, IL-21], or combination thereof. Inhibitors of lysine deacetylase (e.g., TSA) also are incorporated into the T-cell culture at various amounts (e.g., 2.5 ng/ml) and at various times for various durations throughout culturing (e.g., inhibitor introduced at 15 or 30 minutes after start of culture at TO for 24 hours). As a result, these T cells are tuned (or differentiated) into T cells with a specific phenotype (e.g., memory T cell). The tuned T cells are then harvested and the functional phenotype of the harvested T cells is determined. Characterization of cell surface markers or phenotype, metabolic profiling, and/or transcriptional profiling of the harvested T cells determine the functional phenotype of the tune T cells. In some embodiments, the harvested, tuned T cells can be administered based on their functional phenotype to a subject or patient as therapy to produce effective and durable responses to immunotherapy.

The present invention further features an immunotherapeutic method treating a chronic condition in a patient in need thereof, in preferred embodiments, the method comprises first culturing T cells obtained from a source (e.g., patient) and stimulating the T cells in culture with an antigen [(e.g., MHC class 1 molecules), co-stimulatory molecules (e.g., 87-related family members or INF-related family members), cytokines (e.g., IL-1, IL-2, IL-12, IL-21), or combination thereof. Inhibitors of lysine deacetylase (e.g., TSA) also are incorporated into the I-cell culture at various amounts (e.g., 1 nmole to 100 nmoles) and at various times for various durations throughout culturing (e.g., inhibitor introduced at 60 minutes after start of culture at TO for 12 hours. As a result, these T cells are tuned (or differentiated) into T cells with a specific phenotype (e.g., memory T cell for durable immune responses). The tuned T cells are then harvested and the functional phenotype of the harvested T cells is determined. Evaluation of cell surface markers or phenotype, metabolic profiling, and transcriptional profiling of the harvested T cells determines the functional phenotype of the tune T cells. A therapeutic effective amount of said tuned memory T cells is then administered to the patient to produce an effective and durable immunotherapeutic response in the patient.

In preferred embodiments, the functional phenotype of the harvested T cells may comprise memory T cells, commonly characterized as long-lived cells that express a different set of surface markers and respond to antigen with less stringent requirements for activation than do naive T cells. The memory T cell population is generally divided into effector memory (T_{EM}) and central memory (T_{CM}) T cells (FIG. 12). Recently, T memory cells with distinct types of functional attributes have been identified comprising tissue resident memory T cells (T_{RM}), stem cell-like memory T cells (T_{SC}), virtual memory T cells (T_{VM}), and innate memory T cells (T_{IM}), which have unique molecular and functional signatures (FIG. 12).

In preferred circumstances, effective responses to immunotherapy comprise durable or long-lasting (or persistent) responses in absence or presence of continued therapy.

Overall, the present invention features a method using pan lysine deacetylase inhibitors to generate specific memory T cells that, after infusion, would provide lasting effects to reduce the quantity of treatments that patients receive as well as increases the persistency of the treatment. Additionally, this treatment may be engineered to be independent of the pharmaceutical agent, ultimately reducing the overall cost of the treatment. In addition, the present invention allows inhibitors of specific KDAC isoforms (e.g., KDAC1, KDAC2, KDAC6, KDAC11, etc.) to have distinct ability to regulate T cell functional differentiation, and thus can be used to produce a variety of antigen specific functional CD8+ T cells for therapy. Therefore, advantages of this invention include improved personalized medicine, specific memory T cell generation, and lasting treatment for chronic conditions (e.g., cancer and infection) even in the absence of continued therapy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings showing proof of concept using a murine model in which:

FIG. 1. shows in vitro stimulation of naive cells isolated from TCR (I-cell receptor) transgenic mice (OT-1/Rag (h: hour, APO; antigen presenting cell; OCR; oxygen consumption rate; ECAR: extracellular acidification rate).

FIGS. 2A-2B show that KDAC inhibitors regulate antigen induced early activation of CD8+ T cells. KDAC inhibition with TSA at 2.5 ng/ml differentially regulates early activation phenotype of antigen stimulated CD8+ T cells. FIG. 2A shows naïve CD8+ T (OT-1) cells upon Ag stimulation, increase CD69 and decrease CD62L expression in an antigen strength dependent manner, the ratio of antigen bearing microspheres to T cells is indicated (5:1, 1:1 and 1:5). Naïve cells (control) are presented in shaded histograms, and antigen stimulated cells (Ag) are shown by the solid line histogram. FIG. 2B shows that pre-treatment (30 minutes) with TSA (2.5 ng/ml) (shown as dashed line histogram) enhances CD62L loss, but reduces CD69 expression. The results shown are representative of five independent experiments with identical outcomes. (N: Naîve; A; Ag; A+T: Ag+TSA).

FIGS. 3A-3D show that pan KDAC inhibition (e.g., with TSA) reduces TCR proximal signaling in antigen stimulated CD13+ T cells, FIG. 3A shows a schematic of early signaling events in naive CD8+ T cells. FIG. 3B shows a Western blot of cell lysates derived from CD8+ T cells stimulated with antigen either untreated (A) or pre-treated for 30 minutes with TSA (2.5 ng/ml) (A+T) for indicated time points (minutes). Lysates from naïve OT-1 cells served as a control (N). Cell lysates were prepared at 0, 15, 60, or 120 minutes post-stimulation and analyzed for p-LcK^{Tyr394}, p-Zap70, p-PKCe and β-tubulin levels by Western blot analysis. FIG. 3C shows the p-Lck(Tyr394), p-Zap70, p-PKCe levels normalized to β-tubulin (determined by densitometric analysis of Western blot bands via lmageJ software). The normalized values at indicated time-points are plotted. FIG. 3D shows Intracellular staining for pS6K in live gated CD8+ cells. Naïve (shaded histograms, antigen alone (solid line histogram) and antigen plus TSA (histogram indicated by arrow). Error bars are the standard error of mean (SEM) values obtained from three independent experiments. Naïve OT-1 T cells were pre-treated with TSA for 30 minutes and then stimulated with Ag (microspheres bearing H-2K^{b}/cognate 8 amino acid peptide/B7-1), (h: hour: N: Naïve; A: Ag; A+T: Ag+TSA).

FIGS. 4A-40 show that pan KDAC inhibition (ISA) augments asymmetry in antigen stimulated CD+ T cells. FIG. 4A shows dot plots (SSC/FSC) of nave, antigen stimulated (Ag) and TSA pre-treated antigen stimulated (Ag+TSA) CD8+ T cells at 24 h. FIG. 4B shows CD6α expression by live gated lymphocytes at 24 hours (as shown in A), the percentage of low versus high CD8α+ expressing gated T cells is indicated. FIG. 4C shows an overlay histogram of CD8α expression by live gated lymphocytes that were either un-stimulated (Naïve; shaded histogram) or stimulated with antigen alone (Ag; solid line histogram) or pre-treated with TSA and stimulated with antigen (Ag+TSA; dashed line histogram). FIG. 40 shows histograms of pS6K by intracellular staining of live gated CD8 High and CD8 Low cells at 24 h stimulated with antigen in the absence (Ag) or presence of TSA (Ag+TSA). Results shown are representative of three independent experiments with identical outcomes. (h: hour; hi high; lo; low).

FIGS. 5A-5H show that pan KDAC inhibition dampens mTORC1, proliferation and clonal expansion of antigen stimulated CD8+ T cells. Gating strategies and growth profiles of antigen alone (Ag) or TSA induced antigen (Ag+TSA) at 48 hours are shown for CD8 High and CD8 Low populations. Ag and Ag+TSA CD8+ T-cell cultures were FACS sorted on the basis of CD8α expression. FIG. 5A shows the gating strategy of live gated CD8 High and CD8 Low cells in the absence of TSA (Ag) at 48 h, FIG. 5B shows the histograms of pS6K by intracellular staining of live gated CD8 High (top panel) and =CD8 Low (bottom panel) cells stimulated with antigen in the absence of TSA. FIG. 5C shows the overlay histograms of CFSE dye dilution assay of live gated CD8 High (top panel) and CD8 Low (bottom panel) cells stimulated with antigen in the absence of TSA. Solid line histograms represent Ag or Ag+TSA CD8 or High cells and shaded histograms are unstimulated naïve CD8+ T cells. FIG. 5D shows cell numbers (recovered from the culture after 48 h) of CD8+ T cells stimulated with antigen in the absence of TSA. FIG. 5E shows the shows the gating strategy of live gated CD8 High and CD8 Low cells in the presence of TSA (Ag+TSA) at 48 h. FIG. 5F shows the histograms of pS6K by intracellular staining of live gated CD8 High (top panel) and CD8 Low (bottom panel) cells stimulated with antigen in the presence of TSA. FIG. 5G shows the overlay histograms of CFSE dye dilution assay of live gated CD8 High (top panel) and CD8 Low (bottom panel) cells stimulated with antigen in the presence of TSA. Solid line histograms represent Ag or Ag+TSA CD8 Low or High cells and shaded histograms are unstimulated naïve CD8+ T cells. FIG. 5H shows cell numbers (recovered from the culture after 48 h) of CD8+ T cells stimulated with antigen in the presence of TSA. Error bars are the standard error of mean (SEM) values obtained from three independent experiments. (h: hour; hi: high; lo: low).

FIGS. 8A-6H show that pan KDAC inhibition regulates cellular proliferation and clonal expansion of antigen stimulated CD8+ T cells. Pan KDAC inhibition (ISA; 2.5 ng/mol) induced asymmetry reduces clonal expansion of antigen stimulated CD8+ T cells by restricting cell cycle progression and cell division. FIG. 6A shows the sorting strategy of live gated CD8 High and CD8 Low cells stimulated with antigen in the absence (Ag) of TSA at 24 h. TSA pre-treated antigen stimulated CD8+ T cell culture (Ag+TSA) was FAGS sorted on the basis of CD8a expression. These Ag+TSA CD8 Low and CD8 High populations were further cultured for 24 h in the presence of antigen (cell to bead ratio-51) and TSA (2.5 ng/ml). FIG. 6B shows the histograms of pS6K by intracellular staining of CD8 High (top panel) and CD8 Low (bottom panel) cells stimulated with antigen in the absence of TSA. FIG. 6C shows the overlay histograms of CFSE dye dilution assay of live gated CD8 High and CD8 Low cells stimulated with antigen in the absence of TSA. Solid line histograms represent Ag or Ag+TSA CD8 Low or High cells and shaded histograms are unstimulated naïve CD8+ T cells. FIG. 6D shows the cell numbers (recovered from the culture after 72 h) of CD8+ T cells stimulated with antigen in the absence of TSA. FIG. BE shows the gating and sorting strategy of live gated CD8 High and CD8 Low cells stimulated with antigen in the presence of TSA (Ag+TSA) at 24 h. At 24 h. TSA pre-treated antigen stimulated CD8+ T cell (Ag+TSA) culture was FACS sorted on the basis of CD8 expression. These Ag+TSA CD8 Low and CD8 High populations were further cultured for 24 hours in the presence of antigen (cell to bead ratio-5:1) and TSA (2.5 ng/ml). FIG. 6F slows the histograms of pS6K by intraceltular staining of CD8 High and CD8 Low cells stimulated with antigen in the presence of TSA (Ag+TSA). FIG. 6G shows the overlay histograms of CFSE dye dilution assay of live gated CD8 High and CD8 Low cells stimulated with antigen in the or presence of TSA (Ag+TSA). Solid line histograms represent Ag or Ag+TSA CD8 Low or High cells and shaded histograms are unstimulated naïve CD8+ T cells. FIG. 6H shows cell numbers (recovered from the culture after 72 h) of CD8+ T cells stimulated with antigen in the presence of TSA (Ag+TSA). Error bars are the standard error of mean (SEM) values obtained from three independent experiments. (h: hour; hi: high; lo: low).

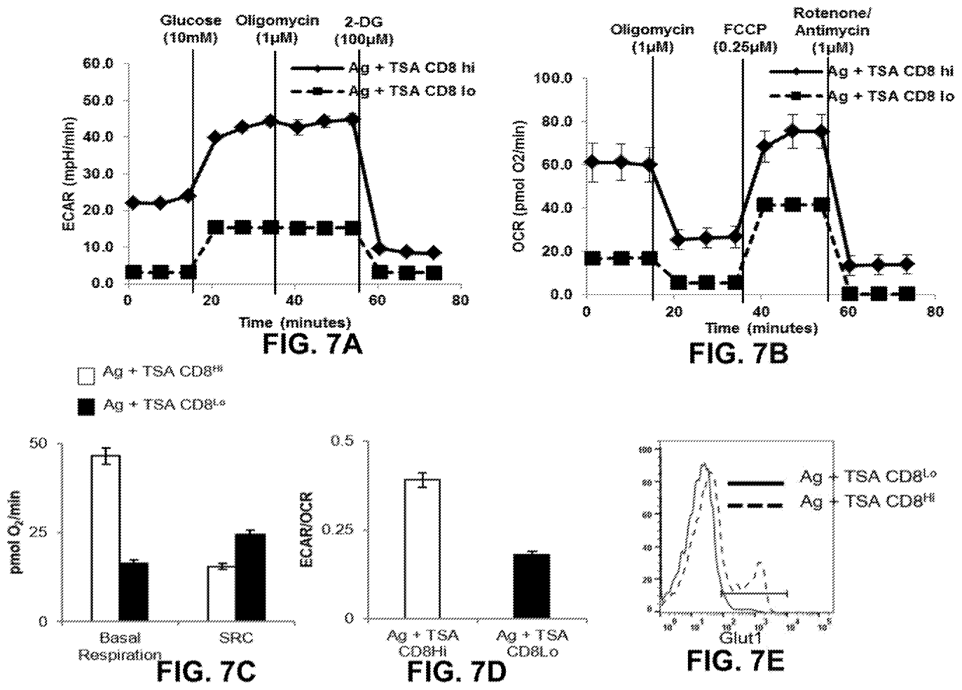

FIGS. 7A-7E show metabolic programming of KDASCi skewed antigen stimulated CD8+ T cells. The metabolic status of TSA induced antigen stimulated asymmetric CD8 High and CD8 Low populations are shown in FIGS. 7A-7D. At 24 h, TSA pre-treated antigen stimulated CD8+ T cells (Ag+TSA) were FAGS sorted on the basis of their CD8a expression and cultured again for another 24 hours (total 48 hours) in the presence of antigen (cell to bead ratio-5:1) and/or TSA (2.5 ng/ml). At indicated time points, the cells were harvested and assayed for glycolysis stress test (extracellular acidification rate; ECAR) and mitochondrial stress test (oxygen consumption rate; OCR). FIG. 7A shows a line graph representing the ECAR test of TSA treated. CD8+ T cells. FIG. 7B shows a line graph representing the OCR test of TSA treated CD8+ T cells. In FIG. 7A and FIG. 7B, the black solid lines represent TSA pre-treated antigen stimulated CD8 High cells (Ag+TSA CCS hi) and the dashed lines represent TSA pre-treated CD8 Low cells (Ag+TSA CD8 lo), FIG. 7C shows a bar graph representing the basal respiration and spare respiratory capacity (SRC). FIG. 7D shows the ECAR to OCR ratio (ECAR/OCR), FIG. 7E shows overlay histograms of staining for glucose transporter 1 (Glutt) expression in TSA pre-treated antigen stimulated CD8 Low cells (Ag+TSA CD8^{lo}; solid line histogram) and CD8 High cells (Ag+TSA CD8^{hi}; dashed line histogram). Error bars are the standard error of mean (SEM) values obtained from three independent experiments (hi: high; lo: low).

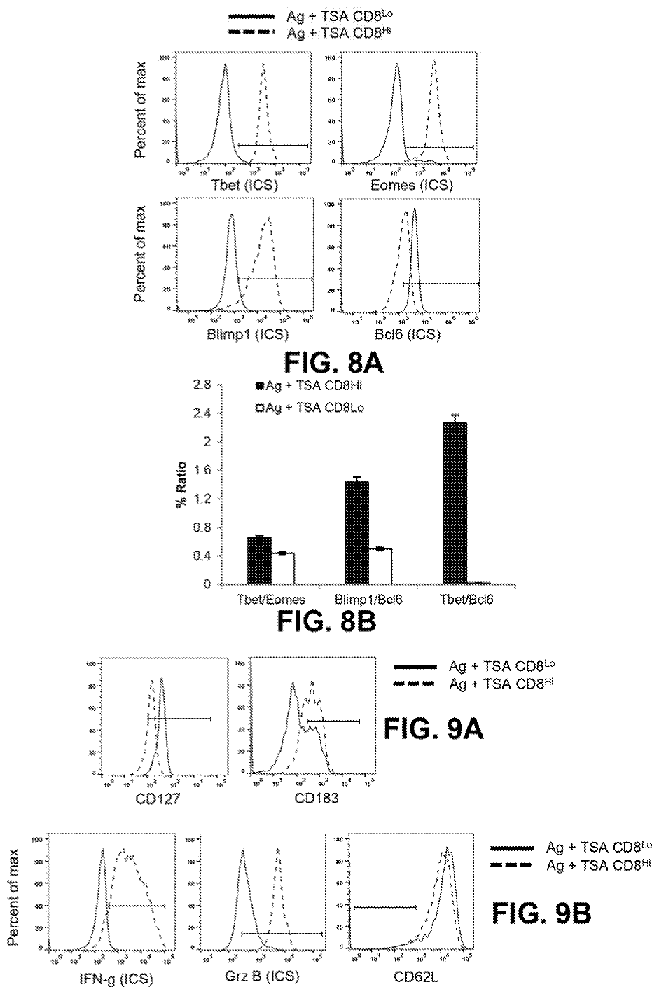

FIGS. 8A-8B show transcriptional characterization of pan KDAC inhibitor treated antigen stimulated CD8+ T cells. The transcriptional characterization of TSA induced antigen stimulated asymmetric CD8 High and CD8 Low populations is shown in FIGS. 8A-8B. At 24 h, TSA pre-treated antigen stimulated CD8+ T cell (Ag+TSA) culture was FACS sorted on the basis of CD8a expression. These Ag+TSA CD8 Low and CD8 High populations were further cultured for 24 hours in the presence of antigen (cell to bead ratio-5:1) and TSA (2.5 ng/ml). Ag+TSA CD8 High and Low cells were permeabilized for intracellular staining of T-bet, Eames, Bcl6 and Blimp1 expression. HG BA shows the histograms for T-bet, Eames, Bcl6 and Blimp1 expression; solid line histograms represent Ag plus TSA CD8 Low cells (Ag+TSA CD8″) and dashed line histograms represent Ag plus TSA CD8 High cells (Ag+TSA CD8) FIG. 86 shows a bar graph representing the ratios of percentage of expression for T-bet. Eomes, Bcl6 and Blimp1 expression. Error bars are the standard error of mean (SEM) values obtained from three independent experiments (h: hour; hi: high; lo: low).

FIGS. 9A-98 show the functional phenotype KDACi treated antigen stimulated CD8+ T cells. The phenotypic characterization is shown for TSA induced antigen stimulated asymmetric CD8 High and CD8 Low populations. At 24 h, TSA pre-treated antigen stimulated CD8+ T cells (Ag+TSA) were FACS sorted on the basis of their CD8α expression and cultured again for another 24 hours (total 48 hours) in the presence of antigen (cell to bead ratio-5:1) and TSA (2.5 ng/ml). FIG. 9A shows the dot plots of CD127 and CD183 double staining of TSA pre-treated antigen stimulated CD8 High cells (Ag+TSA CD8 hi) and CD8 Low cells (Ag+TSA CD8 lo). FIG. 9B shows overlay histograms of TSA pre-treated antigen stimulated CD8 Low cells (Ag+TSA CD8^{lo}; solid line histogram) and CD8 High cells (Ag+TSA CD8^{hi}; dashed line histogram) stained for IFN-g and Granzyme B via intracellular staining and CD62L by surface staining. Results shown are representative of three independent experiments with identical outcomes (hi: high; lo: low).

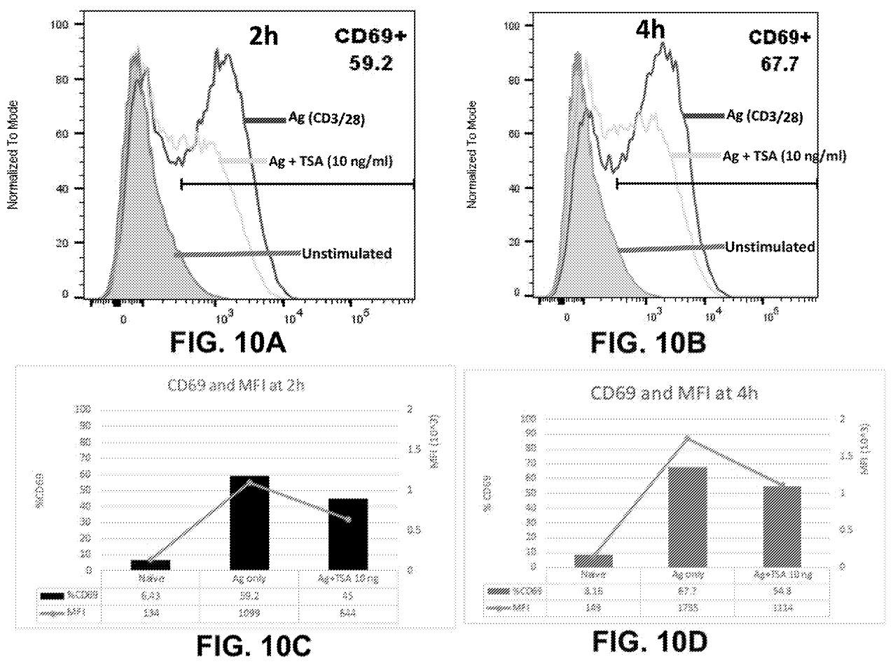

FIGS. 10A-10D show the effect of KDACi on early CD69 expression on Human Jurkat T cells and show that pan KDACi dampens early antigen stimulation of Jurkat T cells. Human Jurkat T Cells were incubated with anti-CD3/anti-0028 (Ag) in the presence or absence of TSA 10 ng/ml) or anti-CD45/CD28 (unstimulated) antibody mated chamber slides for indicated time points. The cells were harvested, counted and stained for CD69. FIGS. 10A and 106 show the overlay histograms demonstrating the difference in the CD69 expression in the presence or absence of TSA at 2 and 4 hours, respectively. FIGS. 10C and 10D show the percentage (% CD69) and their relative Mean Fluorescence intensity (MFI) at 2 and 4 hours, respectively (h: hour).

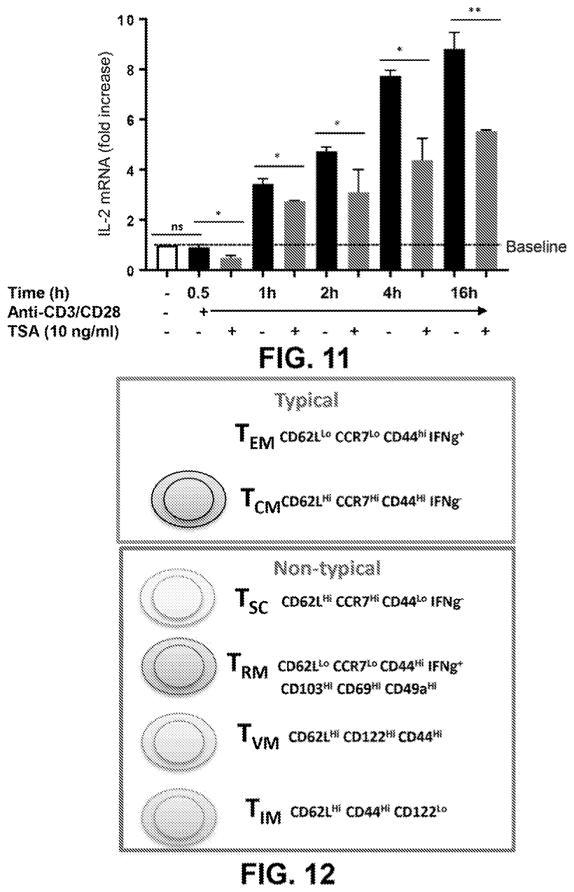

FIG. 11 shows a transcriptional analysis of antigen induced IL-2 gene expression in Human Jurkat cells. IL-2 gene expression is shown in FIG. 11 in stimulated Jurkat T cells in presence or absence of ISA. Cells were incubated on the anti-CD3/anti-CD28 (stimulatory) or anti-CD45 (non-stimulatory) antibody coated chamber slides for 30 min to 16 h, then the total RNA was extracted and performed qPCR analysis of 1L-2 expression. Data shown are the mean fold change of ±SD of three independent experiments. Error bars represent standard deviations. *, statistically significant (p<0.05) and **, p=0.0071 with respect to the values obtained in the presence of TSA. ns, non-significant. (h: hour).

FIG. 12 shows the functional subtypes of memory CDR⁺T cells. Effector memory T cells (T_{w}) are CD62L low (CD62L^{Lo}), C-C chemokine receptor 7 (CCR7^{Lo}), Cluster of Differentiation (CD) 44 high (CD44^{Hi}); and interferon gamma positive (IFNg⁺); T_{CM} cells are CD62L high (CD62L^{H}), CCR7 high (CCR7^{Hi}), CD44^{hi}; and IFNg negative (IFNg⁻); T_{SC} cells are CD62^{Hi}; CCR7^{Hi}, CD44^{Lo}, and IFNg; T_{RM} cells are CD62^{Lo}, CCR7^{Lo}, CD44^{Hi}, CD103 high (CD103^{Hi}), CD69 high (CD69^{Hi}), and CD49a high (CD49a^{Hi}); T_{VM} cells are CD62L^{Hi}, CD122 high (CD122^{Hi}), and CD44^{Hi}; and T_{IM} cells are CD2L^{Hi}, CD44^{Hi}, and CD122^{Lo}, (Hi: high; Lo: low). In preferred embodiments, high and low expression (or negative and positive) refer to expression relative to that normally expressed by nave cells.

### DETAILED DESCRIPTION OF THE INVENTION

As used herein, “administering” and the like refer to the act physically delivering a composition Or other therapy (e g differentiated T cell therapy, immunotherapy) described herein into a subject by such es as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal subcutaneous, intraperitoneal, intraventricular, and intracranial on Radiation therapy can be administered using techniques described herein, including for example, external beam radiation or brachytherapy. When a disease, disorder or condition (e.g., cancer or an infection), or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

As used herein, the terms “re ting” or “treatment” refer to any indicia of success r amelioration of the progression, seventy, and/or duration of a disease, pathology or condition, including any objective or subjective parameter such as abatement; remission; diminishing of symptoms or making the injury; pathology or condition more tolerable to the patient, slowing in the rate of degeneration or decline; making the final point of degeneration less debilitating; or improving a patient's physical or mental well-being.

As used herein, the term “effective amount” as used herein refers to the amount of a therapy (e.g., differentiated T cells or immunotherapy as described herein) which is sufficient to reduce and/or ameliorate the severity and/or duration of a given disease, <disorder or condition and/or a symptom related thereto. This term also encompasses an amount necessary for the reduction or amelioration of the advancement or progression of a given disease (e.g., cancer or infection), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of differentiated T cells or immunotherapy described herein is an amount sufficient to provide a therapeutic benefit in the treatment or management of a cancer or an infection, or to delay or minimize one or mm symptoms associated with the presence of the cancer or an infection. A therapeutically effective amount of an anti-cancer agent described herein, or a radiation therapy described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

As used herein, the term “chronic” refers to “lasting or persisting a long time” or continuing or occurring again and again for a long time. Chronic is a human health condition or disease that is persistent or otherwise long-lasting in its effects or a disease that comes with time. A chronic condition or disease is one that lasts 3 months or more (as per the U.S. National Center for Health Statistics). Chronic diseases are in contrast to those that are acute (abrupt, sharp, and brief) or subacute (within the interval between acute and chronic). Non-limiting examples comprise cancer and long-term infections. Common chronic diseases include arthritis, asthma, cancer, chronic obstructive pulmonary disease, diabetes and some viral diseases such as hepatitis C and acquired immunodeficiency syndrome.

As used herein, the term “tuning” (or tune, tuned) refers to instructing or programming cells or cellular processes for specific differentiation of functions. A iron-limiting example comprises tuning T cells by adding a KDACi at different times and for different durations in culture to instruct or program cells for differentiation into specific functional subtypes of memory T cells, in preferred embodiments, the T cells are tuned early in the T cell activation and/or differentiation process. Tuning also reflects skewing the differentiation of cells to a more particular functional memory T cell that is predominant among a heterogenous population of memory T cells. For example, skewing the differentiation of T cells to comprise 80% central memory T cells and 20% effector memory T cells.

The term “immunotherapy” refers to a treatment of a disease by activating or suppressing the immune system. Immunotherapies designed to elicit or amplify an immune response are classified as activation immunotherapies, while immunotherapies that reduce or suppress are classified as suppression immunotherapies. Immunotherapy is a type of therapy that uses substances to stimulate or suppress the immune system to help the body fight cancer, infection, and other diseases. Some types of immunotherapy only target certain cells of the immune system. Others affect the immune system in a general way. Types of immunotherapy include cytokines, vaccines, bacillus Calmette-Guerin (BCG), and some monoclonal antibodies, immunotherapy uses the body's immune system to fight cancer. Non-limiting ex ivies of three types of immunotherapy used to treat sneer comprise nonspecific immune stimulation T-cell transfer therapy (CART; engineered T cells), and immune checkpoint inhibitors.

The term “cancer” refers to any physiological condition in mammals characterized by unregulated cell growth. Cancers described herein include solid tumors and hematological (blood) cancers, A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “anti-cancer agent” is used accordance with its plain ordinary meaning and refers to a composition having anti-neoplastic properties or the ability to Inhibit the growth or proliferation of cells. In certain embodiments, an anti-cancer agent is a chemotherapeutic. In certain embodiments, an anti-cancer agent is an agent identified herein having utility in methods of treating cancer. In certain embodiments, an anti-cancer agent is an agent approved by the FDA or similar regulatory agency of a country other than the USA, for treating cancer.

The term ‘anti-microbial agent’ is used in accordance with its plain ordinary meaning and refers to a composition having anti-bacterial, anti-viral, and/or anti-parasitic properties. A non-limiting example of an anti-microbial agent comprises antibiotics, which include, but are not limited to, penicillins, tetracyclines, cephalosporins, quinolones, lincomycins, macrolides, sulronamides, glycopeptide antibiotics, aminoglycosides, carbapenems; ansamycins, lipopeptides, monobactams, nitrofurans, oxaxoliclinones, and polypeptides.

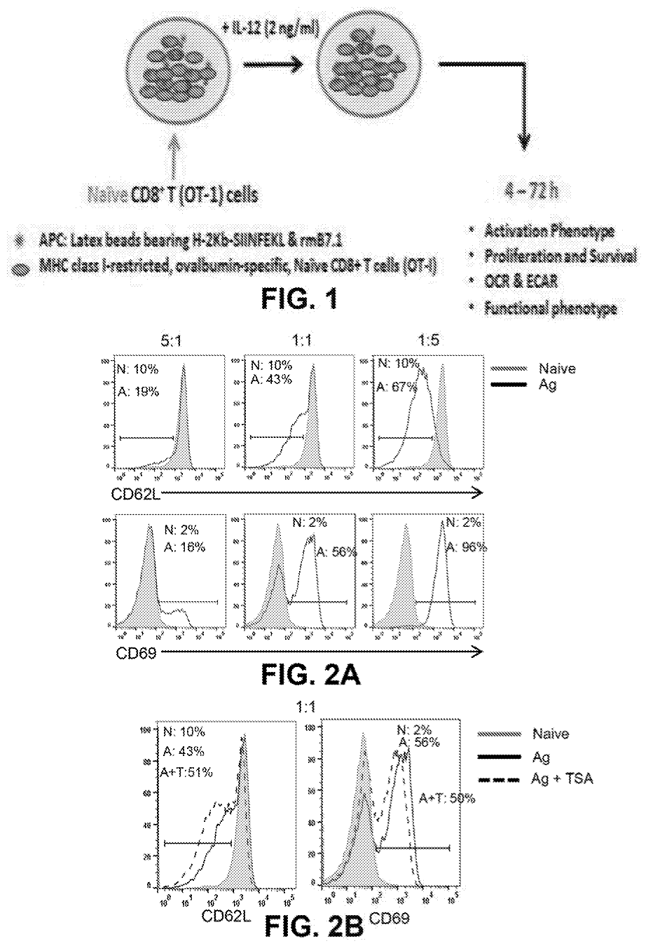

Referring now to FIGS. 1-12, the present invention features a method for using pan KDAC inhibition during T-cell culture to tune their differentiation into memory T cells for persistent antigen specific responses. The memory cells are characterized by their cell surface phenotype, metabolic profile, transcription/signaling profile, and their functional phenotype. This method is useful to generate a specific memory T cell population that is more effective in T cell therapy of chronic challenges such as cancer and/or infections.

In preferred embodiments, the present invention features a method to generate specific memory T cells that, after infusion, would provide lasting effects (e.g., to produce durable response) to reduce the quantity of treatments that patients receive as well as increases the persistency of the treatment.

Relevant applications of this technology comprise; 1) durable immunotherapy generation for the pharmaceutical industry; 2) patient-specific immunotherapy for personalized medicine; and 3) specific memory T cell population generation or T cell therapy for cancer and/or infections for cancer immunotherapy.

Relevant advantages of this technology comprise 1 More effective T cell therapy for chronic challenges (i.e. cancer and/or infections) 2) personalized treatment; specific memory T cell generation; and 3) lasting treaty chronic conditions (i.e. cancer and infection) (cheaper).

The present invention features methods of introducing KDAC inhibitors to the culture of non-stimulated or stimulated T cells (e.g., during the process of CAR T cell generation or engineered T cell generation) at various amounts and at various times and durations of culture to tune their differentiation into T cells with specific functional phenotypes for persistent antigen specific responses. The tuned cells can then be harvested, functionally characterized, and administered to subjects for effective immunotherapy responses. This unique approach of the present invention allows for personalized immunotherapy development across a wide variety of immunotherapeutic platforms.

In preferred embodiments, the source of T cells may comprise human subjects and/or cell culture. The T cell population may comprise T cells of various lineages.

In preferred embodiments, antigen stimulation of T-cell culture occurs at time 0 (TO), in some embodiments, the antigen stimulation comprises stimulating with one or more of the following: antigens; co-stimulatory molecules; and cytokines.

A non-limiting example of an antigen for stimulating the T cells comprises major histocompatibility complex (MHC) Class I (HLA-A, B, or C) molecules bearing cognate tumor antigen or self-antigen (Ag), which can be immobilized on in vitro latex microspheres. In some embodiments, the amount of antigen ranges from 0.1 nmoles to 1000 nmoles, and in preferred embodiments, the amount is 10 nmoles.

Non-limiting examples of co-stimulatory molecules comprise B7-related family members and/or or TNF-related family members. The concentration range of the co-stimulatory molecules comprises from about 0.1 ng/ml to about 2000 ng/ml; and in preferred embodiments, the concentration is 1000 ng/ml.

Non-limiting examples of cytokine comprise IL-1, IL-2, IL-12, and/or IL-21. In some embodiments, the cytokine concentration ranges from about 02 ng/ml to about 200 ng/ml.

In some embodiments, memory T cell responses are persistent and demonstrate ideal characteristics (e.g., to produce durable and long-lasting responses) for chronic challenges including but not limited to cancer and chronic infections.

Histone deacetylase (HDAC) proteins are now called lysine deacetylase proteins (KDAC), to describe their function rather than their target which, also includes non-histone proteins. In some embodiments, the inhibitors comprise first generation KDAC inhibitors including but not limited to hydroxamic acids (or hydroxamates), such as TSA, cyclic tetrapeptides (such as trapoxin B), and the depsipeptides, benzamides, electrophilic ketones, and the aliphatic acid compounds such as phenylbutyrate and valproic acid.

In other embodiments, KDAC inhibitors comprise second-generation inhibitors comprising the hydroxamic acids vorinostat (SAHA), belinostat (PXD101), LAQ824, and panobinostat (LBH580); and the benzamides: entinostat (MS-275), tacedinaline (C1994), and mocetinostat (MGCD0103). The sirtuin Class III HDACs are dependent on NAD+ and are, therefore, inhibited by nicotinamide, as well as derivatives of NAD, dihydrocoumarin, naphthopyranone, and 2-hydroxynaphthaldehydes. In some embodiments, KDAC inhibitors include third generation inhibitors comprising OSU-HDAG42.

In some embodiments, the amount of KDAC inhibitors ranges from about 1 nmole to about 100 nmoles. A non-limiting example comprises administering TSA at 2.5 ng/real.

In appropriate circumstances, the KDAC inhibitors are introduced at various times of culture to induce differential T cell functional phenotype. Non-limiting examples of the time of KDACi introduction to the culture comprise T0−24 hours, T0−60 minutes, T0−30 minutes, T0. T0+30 minutes, T0−60 minutes, up to T0+24 hours, wherein T0 is the time of T cell stimulation.

In additional circumstances KDAC inhibitors are introduced for varying durations to induce differential T cell functional phenotype. Non-limiting examples of the duration of KDAC inhibition comprises up to about 2 hours, up to about 6 hours, up to about 12 hours, up to about 24 hours.

In some embodiments, the method features harvesting the cells at different times. Non-limiting examples comprise from about 24 to about 72 hours from TO and cells can be subjected to re-stimulation multiple times.

In some embodiments, the present invention comprises a method that features the introduction of a KDAC inhibitor during T-cell culture and/or vaccination to tune T cell differentiation towards memory T cells for persistent antigen specific responses. In preferred embodiments, the methods feature determining the functional phenotype of cultured T cells by their surface phenotype, metabolic profile, and/or transcription/signaling profile.

A non-limiting example comprises KDAC inhibition differentially regulating antigen dose-dependent T cell proximal signaling CD8+ T cell activation; KDAC inhibition reduces T cell proximal TCR signaling and mTORC1/2 activity. Another non-limiting example comprises pan KDAC inhibition enhances the induction of asymmetry in CD8+ T cells (prior to cell division); TSA induces antigen stimulated asymmetric CD8 High and CD8 Low populations.

The present invention further features a method that differentially regulates antigen induced early T cell activation phenotype by selectively producing a functional phenotype with distinct surface markers and transcriptional profiles. Non-limiting examples comprise generating: 1) an effector memory T cell population with low CD62L, low CCR7, and high CD44 expression and positive for IFNg; 2) a central memory T cell population with high CD62L, high CCR7, and CD44 high expression and negative for IFNg; 3) a stem cell-like memory T cell population with high CD62I, high CCR7, and low CD44 expression and negative for IFNg; 4) a resident memory T cell population with CD62L low, CCR7 low, high CD44, high CD103, high CD69, and high CD49a expression and positive for IFNg; 5) a virtual memory T cell population with high CD62L, high CD122, and high CD44 expression; and 6) an innate memory T cell population with high CD62L, high CD44, and low CD122 expression.

In some embodiments, the present invention features a method that produces a specific memory T cell population for personalized treatment.

In some embodiments, pre-treating T-cell cultures with KDAC inhibitors reduces dose-dependent CD69 expression and increase CD62L shedding. A non-limiting example comprises pan KDAC inhibition differentially regulating antigen induced early T− cell activation phenotype by selectively restricting antigen stimulation induced CD69 transcription but enhancing CD62L shedding.

In appropriate circumstances, the method can be utilized to produce a specific memory T cell population for treatment, wherein the cultured cells are then re-administered into the patient for treatment. In preferred embodiments, the method allows use of KDAC inhibitors that are specific for specific KDAC isoforms to regulate T cell functional differentiation and produce distinct antigen specific functional CD8+ T cells for therapy.

### EXAMPLES

The following are non-limiting examples of practicing the present invention. It is to be understood that the invention is not limited to the examples described herein. Equivalents or substitutes are within the scope of the invention.

Examples 1-0 were obtained from a murine transgenic model, from which in vitro stimulated naïve cells were isolated from TCR transgenic mice (OT-1/Rag -/-). In brief, the CD8+ T cells obtained from nave TCR transgenic mice (OT-1/Rag -/-) mice were stimulated in vitro with latex microspheres on which major histocompatibility complex (MHC) Class I (H-2Kb) dimers bearing 10 nM of cognate peptide (Ag) were immobilized, along with 1 μg/ml of recombinant murine 87.1 (co-stimulation) and 2 ng/ml of rmIL-12 (cytokine) (FIG. 1).

Examples 9-10 were obtained from human Jurkat cell line. Jurkat T cells were stimulated in vitro with latex microspheres on which major histocompatibility complex (MHC) Class I (H-2Kb) dimers bearing 10 nM of cognate peptide (Ag) were immobilized, along with 1 μg/ml of recombinant murine 87.1 (co-stimulation) and 2 ng/ml of rmIL-12 (cytokine)

### EXAMPLE 1: KDAC Inhibitors Regulate Antigen-Induced Early Activation of CD8+ T Cells

Cognate antigen presented by MHC Class 1 to irate CD8+ T cells in the context of co-stimulation and cytokine leads to rapid (2-4 hours) increases in CD69 expression and decreases in CD62L, which indicate antigen induced early T cell activation response. To characterize early activation of T cells, naïve CD8+(OT-I) T cells were reacted with antigen (Ag; latex microspheres bearing H-2Kb-Fc+/8 amoni acid cognate peptide/rmB7-1) in vitro for 4 hours, cell surface was stained for CD82L and CD69 expression and evaluated by flow cytometry. The results in FIG. 2 indicate that down-regulation of CD62L, which is due to proteolytic shedding, and up-regulation of CD69, which is due to de novo transcription, occur in an antigen strength (dose) dependent manner (cell to bead ratio-5:1, 1:1 and 1:5, altered peptides produce identical outcomes; data not shown). Strikingly, pre-treatment with the pan KDAC inhibitor, TSA (2.5 ng/ml) reduces antigen dose dependent CD69 expression but increases CD62L shedding (TIMP inhibition data). Thus, pan KDAC inhibition differentially regulates antigen induced early T cell activation phenotype by selectively restricting antigen, stimulation-induced CD69 transcription but enhancing CD62L shedding.

### EXAMPLE 2: Pan KOAC Inhibition Reduces TCR Proximal Signaling in Antigen Stimulated CD8+ T Cells

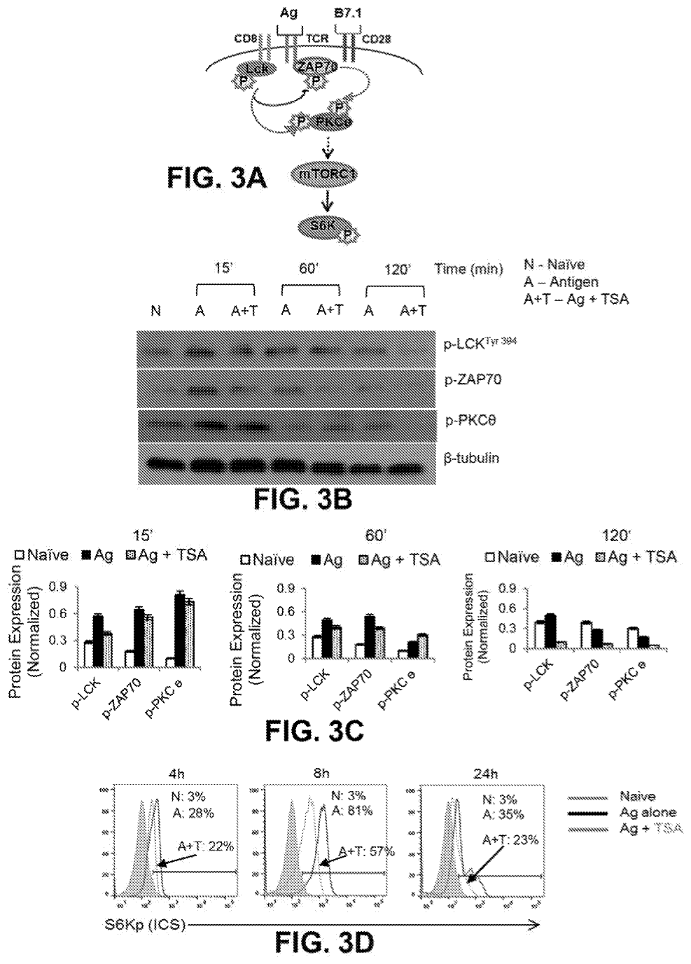

As antigen stimulated CD69 induction has been shown to require PKCθ phosphorylation, the reduction of CD69 expression by KDACi most likely occurs due to dampened early antigen-mediated TCR signaling events. FIG. 3A shows a schematic of early signaling events in naive CD8+ T cells. Western blot analysis was conducted to compare the level of phosphorylation of early TCR signaling proteins, Lck, Zap70, and PKCθ, by TSA pre-treatment of antigen stimulated CD8+ T cells. As shown in FIGS. 3B and 3C, phosphorylation of Lck is dampened by 15 minutes followed by reduction of Zap70 as well as PKCθ in ISA pretreated Ag stimulated CD8+ T cells (Ag+TSA; A+T) compared to the cells treated with antigen alone (Ag alone; A); sequential phosphorylation of Lck, Zap70 and PKCe at 15, 60 and 120 minutes is kinetically dampened by TSA pre-treatment. Since, the energy sensitive kinase mTORC1 serves as an integrative node for extracellular signals that initiate naïve CD8+ T cells activation, the mTORC1 activity was assessed by measuring the phosphorylation state of the mTORC1 target ribosomal 86 (p-S6) by intracellular flow cytometry (FIG. 3D). FIG. 3D shows that intracellular staining for pS6K ire CD8+ T cells demonstrates lower mTORC1 activity by flow cytometry; S6Kp was lower at both 4 and 8 hours post-antigen stimulation of naïve OT-1 T cells indicating that the pretreatment with TSA dampened mTORC1 activity.

### EXAMPLE 3: Pan KDAC Inhibition Augments Asymmetry in Antigen Stimulated CD8+ T Cells

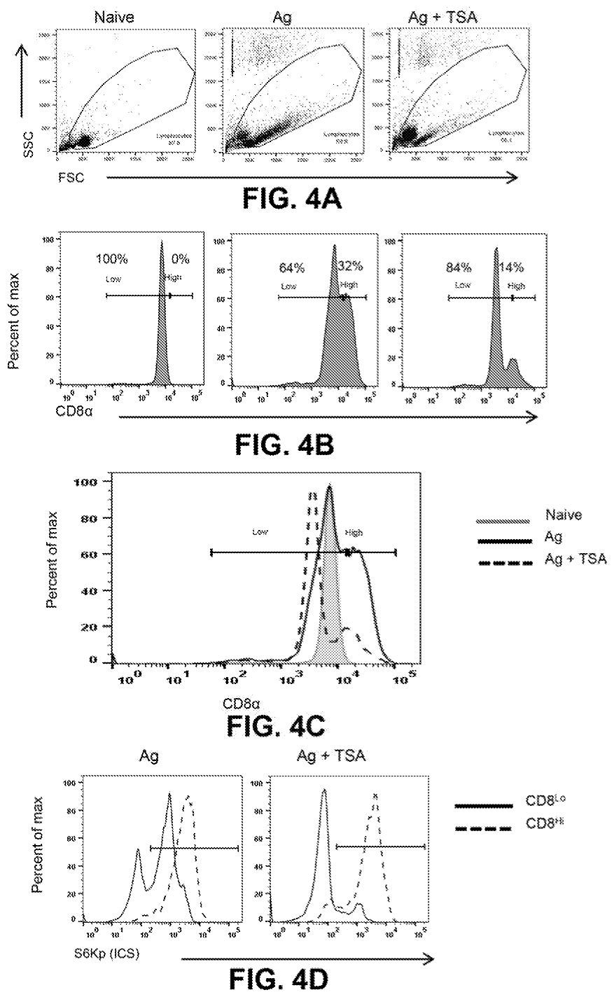

Accumulating evidence implicates a deterministic role for cellular asymmetry in antigen induced CD8+ T cells division and functional maturation. The KDACi-mediated reduced TCR signaling and mTOR activity were examined to determine their effect on the induction of inherent asymmetry produced in antigen stimulated CD8+ T cells (FIGS. 4A-4D). It was initially observed that upon activation, within 24 h, CD8 expression is up-regulated in a certain percentage of cells. Surprisingly, TSA pre-treated antigen stimulated CD8+ T cells had tuned CD8 expression as compared to antigen alone. TSA renders a lesser proportion of cells to have increased expression of CD8 as compared to Ag alone. This could be attributed to the lower threshold of early activation/TCR signaling cues available to the TSA primed CD8+ T cells.

### EXAMPLE 4: Pan KOAC Inhibition Dampens mTORC1, Proliferation and Clonal Expansion of Antigen Stimulated CD8+ T Cells

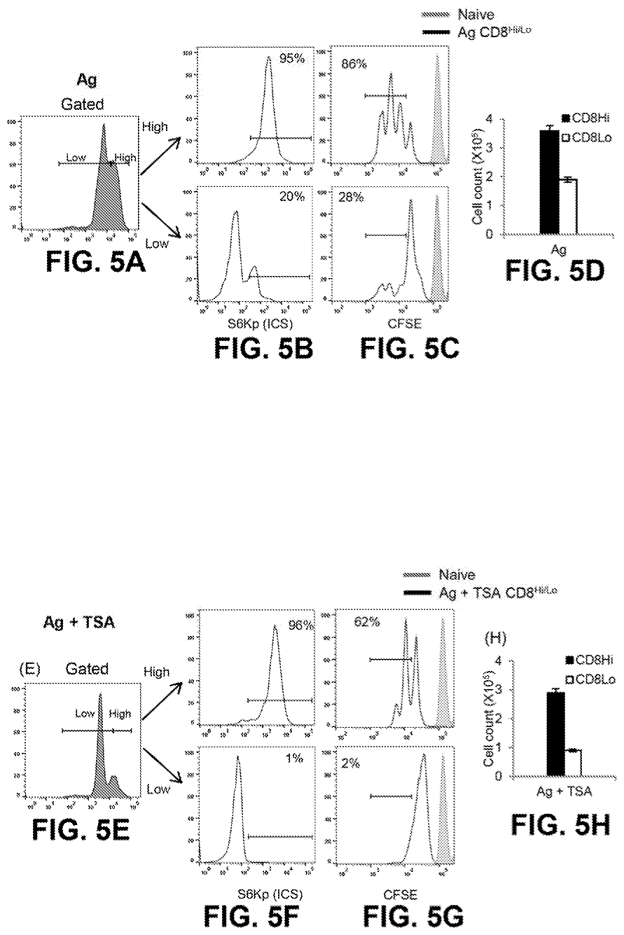

To determine the implications of the TSA-mediated tuned percentage of CD8 expression on the growth profile, of these cells, the 24 hours Ag and Ag # TSA CD8+ T cells were separated by FACS sorting on the basis of their CD8 expression (FIGS. 5A-5H). Further, these CUB Low and CD8 High cells Mere cultured separately in the presence of Ag and or TSA for 24 hours (total 48 hours). mTORC1 activity (represented by S6Kp) was higher in TSA primed Ag stimulated CD8 Hi (FIG. 5F, top panel) cells as compared to Ag alone CD8 Hi cells (FIG. 5B, top panel), whereas the Ag+TSA CD8 Low cells (FIG. 5F, bottom panel) had a reduced S6Kp as compared to Ag CD8 Low cells (FIG. 5B, bottom panel). Since mTORC1 activity affects growth, cell cycle, and proliferation, Ag+TSA CD8 Low cells are believed to be retained in the G0-G1 phase of cell cycle and do not enter the S phase, whereas Ag CD8 Low cells follow the same trend but are able to enter the S phase (FIG. 5D). In contrast, significant percentage of Ag+TSA CD8 High enter S phase, comparable to the Ag CD8 Hi cells FIG. 5H) SE dye dilution assay also shows the same trend of cell division (FIGS. 5C and 5G).

### EXAMPLE 5: KDAC Inhibition Regulates Cellular Proliferation and Clonal Expansion of Antigen-Stimulated CD8+ T Cells

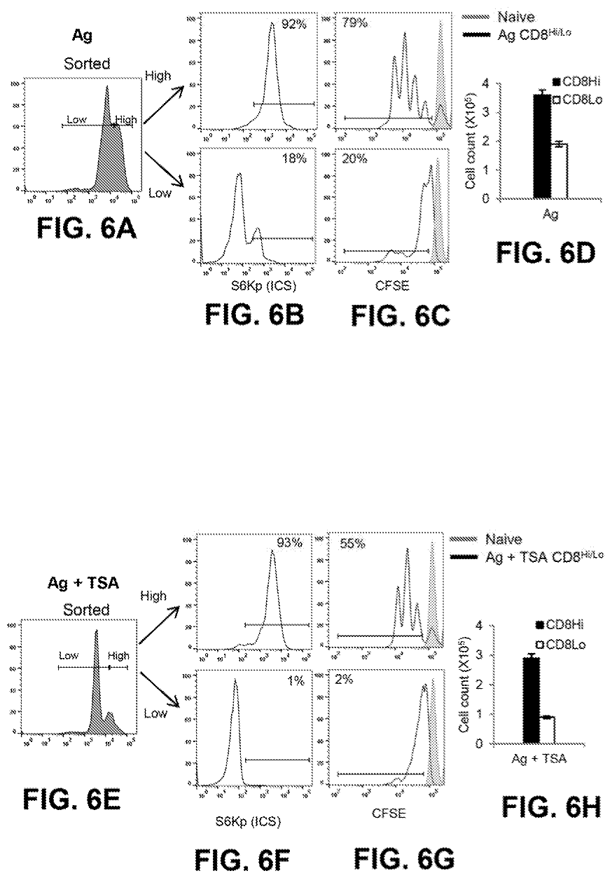

FIGS. 6A-8H show that pan KDAC inhibition (TSA, 2.5 ng/ml)-induced asymmetry reduces clonal expansion of antigen stimulated CD8+ T cells by restricting, cell cycle progression and cell division.

### EXAMPLE 6: Metabolic Programming of KDAC Inhibition Skewed Antigen Stimulated CD8+ T Cells

It was recently shown that asymmetric partitioning of mTORC1 activity upon activation of naïve CD8+ T cells results in the generation of two nascent daughter cells with different metabolic profiles for fate determination. TSA-mediated asymmetric CD8 Low and Hi cells also have distinct metabolic profiles as shown in FIGS. 7A-7E. Glycolysis (ECAR) (FIG. 7A) and mitochondrial (OCR) (FIG. 7B) stress tests show that Ag+TSA CD8 Hi cells have higher ECAR as well as OCR as compared to Ag+TSA CD8 Low cells. The high cells have higher dependence on ECAR as compared to the low cells as demonstrated by the ECAR/OCR ratio and also their Glut1 expression. Spare respiratory capacity (SRC) has been linked with memory like cells and SRC is higher in Ag+TSA Low cells as compared to the Ag+TSA Hi cells (FIG. 7C). FIG. 70 shows the ECAR to OCR ratio (ECAR/OCR).

### EXAMPLE 7: Transcriptional Characterization of TSA-Induced, Antigen Stimulated Asymmetric CD8 High and CD8 Low Populations

Because of the evident differences in the mTOR activity and growth profile of the Ag+TSA CD8 and high cells, their transcriptional profile was further investigated. Tbet/Eomes, Blimp1/Bcl6 and Tbet/Bcl6 ratios are typically used to characterize the effector versus memory like status of CD8+ T cells. The 48 hours (24 hours sort+24 hours culture) Ag+TSA CIO Low (Lo) cells were observed to have significantly reduced Tbet/Eomes, Blimp1/Bcl6 and Tbet/Bcl6 ratios (FIGS. 8A-8B), clearly suggesting their memory like status.

### EXAMPLE 8: Functional Phenotype of TSA-Induced, Antigen Stimulated Asymmetric CD8 High and CD8 Low Populations

To further confirm the memory precursor status of the Ag+TSA CD8 Low cells, the expression of various phenotypic markers, associated with functional maturation was determined; effector and/or memory (FIGS. 9A-9B). When comparing these populations, increased expression of memory precursor markers including CD127 and CD62L in Ag+TSA CD8 Low cells was observed compared to Ag+TSA CD8 High cells, in agreement, the effector molecules including IFN-g, granzyme B and CD183 were higher Ag+TSA CD8 Hi cells as compared to Ag+TSA CD8 Low cells. These observations confirm that KDACi pretreatment induces or tunes CD8 expression asymmetry to predict their subsequent development into functionally distinct phenotype by regulating transcriptional metabolic profiles as compared to the effector Ag+TSA CD8 High cells.

### EXAMPLE 9: Effect of KDAC Inhibition on Early CD69 Expression on (Human) Jurkat T Cells

FIGS. 10A-10D shows that pan KDAC inhibition reduces early antigen stimulation of Jurkat T cells.

### EXAMPLE 10: Transcriptional Analysis of Antigen-Induced IL-2 Gene Expression in Human Jurkat T Cells

FIG. 11 shows that pan KDAC inhibition reduces IL-2 gene expression in stimulated Jurkat T cells in the presence of TSA.

### EXAMPLE 11: EMBODIMENTS OF NON-LIMITING FUNCTIONAL SUBTYPES OF MEMORY CD8+ T CELLS

FIG. 12 shows embodiments of the present invention differentially resulting in functional subtypes of memory T cells. Non-limiting examples of functional phenotypes for: 1) effector memory T cell comprises CD62L low, CCR7 low. CD44 high, and IFNg positive; 2) central memory T cell comprises CD62L high, CCR7 high, CD44 high, and IFNg negative; 3) stem cell-like memory T cell comprises CD62I high, CCR7 high, CD44 low, and IFNg negative; 4) resident memory T cell comprises CD62t, low, CCR7 low, CD44 high, IFNg positive, CD103 high, CD69 high, and CD49a high; 5) virtual memory T cell comprises CD62L high, CD122 high, and CD44 high; and 6) innate memory T cell comprising CD62L high, CD44 high, and CD122 low.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent, office only, and are not limiting in any way, in some embodiments, the figures presented in this patent application are drawn to scale, including the angles, ratios of dimensions, etc. In some embodiments, the figures are representative only and the claims are not limited by the dimensions of the figures. In some embodiments, descriptions ref the inventions described herein using the phrase “comprising” includes embodiments that could be described as “consisting of”, and as such the written description requirement for claiming one or more embodiments of the present invention using the phrase “consisting of” is met.

1. An in vitro method of tuning T cells to generate a population of T cells with a specific functional phenotype for a durable immunotherapy response, said method comprising:
a. culturing said T cells obtained from a source;
b. stimulating said T cells in culture with antigen(s), co-stimulatory molecule(s), cytokine(s), or combination thereof;
c. incorporating an inhibitor of lysine deacetylase (KDACi) to said T-cell culture at various amounts of said inhibitor, at various times of culture, and for various durations of culture;
d. harvesting said cultured T cells;
e. determining said functional phenotype of said cultured T cells,
wherein said specific functional phenotype of tuned differentiated T cells is memory T cells that produce said durable immunotherapy response.

2. An immunotherapeutic method of treating a chronic condition in a patient in need thereof, said method comprises:
a. culturing T cells obtained from a source;
b. stimulating said T cells in culture with antigen(s), co-stimulatory molecule(s), cytokine(s), or combination thereof;
c. incorporating an inhibitor of lysine deacetylase (KDACi) to said T-cell culture at various amounts of said inhibitor, at various times of culture, and for various durations of culture;
d. harvesting said cultured T cells;
e. determining a functional phenotype of said cultured T cells,
wherein said functional phenotype of said cultured T cells is differentiated or tuned memory T cells that produce a durable immunotherapy response; and
f. administering a therapeutic effective amount of said tuned memory T cells to said patient,
wherein said tuned memory T cells produce said durable immunotherapeutic response in said patient.

3. The method of claim 1, wherein said source of T cells comprises a human subject and/or cell culture.

4. The method of claim 1, wherein stimulating said T cells in culture occurs at time TO.

5. The method of claim 1, wherein the antigen for stimulating said T cells comprises major histocompatibility complex (MHC) Class I (HLA-A, B, or C) molecules bearing cognate tumor antigen or self-antigen, which can be immobilized on in vitro latex microspheres.

6. (canceled)

7. The method of claim 1, wherein said co-stimulatory molecules comprise B7-related family members and/or or TNF-related family members.

8. (canceled)

9. The method of claim 1, wherein said cytokine comprises IL-1, IL-2, IL-12, and/or IL-21.

10. (canceled)

11. The method of claim 1, wherein said inhibitors of KDAC activity comprise molecules that inhibit enzymes that de-acetylate lysine amino acids, wherein said molecules comprise trichostatin A (TSA), suberoylanilide hydroxamic acid (SAHA; vorinostat), sodium butyrate, oxamflatin, scriptaid (N-Hydroxy-1,3-dioxo-1H-benz(de)isoquinoline-2(3H)-hexan amide), panobinostat, romidepsin, and valproic acid.

12. The method of claim 11, wherein an amount of said KDAC inhibitors ranges from about 1 nmole to about 100 nmoles, wherein said amount is effective at inhibiting KDAC activity in culture.

13. The method of claim 11, wherein said KDAC inhibitors are introduced at various times of culture to induce differential T cell functional phenotype.

14. The method of claim 13, wherein said time of introduction of KDAC inhibitors to said culture comprises T0−24 hours, T0−60 minutes, T0−30 minutes, T0, T0+30 minutes, T0+60 minutes, or up to T0+24 hours, wherein T0 is initial time of T cell stimulation.

15. The method of claim 11, wherein said KDAC inhibitors are introduced for varying durations to induce differential T cell functional phenotype.

16. The method of claim 15, wherein said duration of KDAC inhibition comprises up to about 2 hours, up to 6 about hours, up to about 12 hours, or up to about 24 hours.

17. The method of claim 1, wherein harvesting said T cells occurs at various times of said culture ranging from about 24 to 72 hours from T0.

18. (canceled)

19. The method of claim 1, wherein cell surface marker(s), functional phenotype marker(s), metabolic factor(s), and/or transcriptional factor(s) are used to characterize differential functional phenotype of said T cells.

20. The method of claim 19, wherein said cell surface markers of T cells comprise CD8, CD44, CD49a, CD62L, CD69, CD122, CD127, and/or CD183.

21. (canceled)

22. The method of claim 19, wherein functional phenotype marker(s) comprise CD44, CD49a, CD62L, CD69, CD122CD127, CD183, IFN-g, and/or Granzyme B.

23. The method of claim 19, wherein said metabolic factors comprise glycolysis stress test factors (ECAR), mitochondrial stress test factors (OCR), ECAR/OCR ratio, GLUT1 expression, and/or spare respiratory capacity.

24. The method of claim 19, wherein said transcription factors comprise T-bet, Eomes, BcI6, Blimp1; Tbet/Eomes ratio, Blimp1/Bcl6 ratio and/or Tbet/Bcl6 ratio.

25. The method of claim 1, wherein said KDAC inhibitor is introduced during T-cell culture and/or vaccination to tune T cell differentiation towards memory T cells for persistent antigen specific responses.

26.-43. (canceled)
